(19) United States
(12) Patent Application Publication
(10) Pub. No.: US 2026/0198939 A1
(43) Pub. Date: Jul. 16, 2026

(54) Rapid Distal Locking Jig

(71) Applicant: Mohammed Imran Alam, Milton Keynes (GB)

(72) Inventor: Mohammed Imran Alam, Milton Keynes (GB)

(21) Appl. No.: 19/016,382

(22) Filed: Jan. 10, 2025

(51) Int. Cl. A61B 17/17; A61B 17/72

(52) U.S. Cl. CPC A61B 17/1725; A61B 17/7233

(57) ABSTRACT

The present invention relates to a rapid distal locking jig which is configured to improve the precision and efficiency of screw insertion in femoral nail fixation surgery. The jig consists of an interconnecting bar linking proximal and distal targeting arms, ensuring alignment with pre-drilled holes in the femoral nail for accurate screw insertion. The jig includes sleeves which help in aligning its parallel targeting arms with the drilled holes and in insertion of screws, with innermost of the 3 sleeve occupying the drilled holes spaces and the spaces between the innermost and outermost sleeve are filled with the spacer sleeves. A guide wire stabilizes the jig preventing tilting and ensuring correct placement. The invention involves aligning the nail holes with a C-arm, inserting guide wires and drilling precise holes for screw insertion. An alignment guide with a ring and arrowhead extensions further ensures alignment of the screw holes.

### TECHNICAL FIELD

The invention relates to the field of orthopaedic surgical tools, more specifically to a rapid distal locking jig for locking of distal holes of intramedullary nail with screws. The tool is configured to improve the efficiency and accuracy of distal screw insertion in femoral nailing procedure, minimizing the need for X-ray imaging and reducing the risk of bone damage and radiation exposure.

### BACKGROUND

The background description includes information that may be useful in understanding the present invention. It is not an admission that any of the information provided herein is prior art or relevant to the presently claimed invention, or that any publication specifically or implicitly referenced is prior art.

The intramedullary nailing (IM nailing) is a well-established and widely used method for stabilizing fractures of the femur. The method involves the insertion of a metal nail into the medullary canal of the femur which serves as an internal splint to stabilize the bone. After proper positioning of the nail both ends of the nail are secured to the bone with the use of locking screws. The locking screws are inserted through holes located at both the proximal and distal ends of the intramedullary nail to ensure proper fixation and stabilization.

Traditionally, the insertion of locking screws through the distal end of the nail is performed using the free-hand technique. The technique involves manual drilling and screw insertion without the assistance of advanced guidance tools making it highly dependent on the surgeon's skill and experience. During the process, the surgeon must rely on their ability to position the drill and screw manually, often using visual cues and palpation to aim. While the technique is widely practiced, still it is far from optimal.

The free-hand technique is heavily reliant on the use of X-rays for guidance which introduces several inherent challenges. In the traditional method, the surgeon must attempt to position the drill or screw in the desired direction through a process of trial and error. The Hit and Trial approach can result in multiple failed attempts, each requiring X-ray imaging to confirm the alignment of the drill bit and the screw. The X-ray exposure after each attempt significantly increases the overall time required for the procedure and subjects both the surgical team and the patient to unnecessary radiation. Furthermore, X-ray imaging is limited in ability to clearly capture the movement of the drill bit and screw, especially when the surgeon's hands are in the line of the X-ray beam. The limitation prevents accurate real-time feedback during the procedure.

Another significant issue with the free-hand technique is that each failed attempt to drill or insert a screw creates a false hole in the bone, particularly in the medial cortex of the distal femur. The false holes compromise the structural integrity of the bone, potentially leading to weakened areas of bone that may complicate future recovery. The accumulation of the errors further prolongs the procedure and increases the risk of complications, particularly for patients with fragile bone structures or in the hands of less experienced surgeons.

The pressing need for a more efficient, precise and less radiation-dependent method for distal locking screw insertion have led to the development of several advanced jigs and devices. Some of the innovations include the Trigen (Smith & Nephew) distal locking device known as SureShot which utilizes a highly advanced electromagnetic field tracking system to guide the insertion of locking screws. The device involves the use of a non-magnetic drill and a sensor probe that generates a virtual image of the distal nail and screw hole. Although the device can enhance precision, but it requires a well-trained team and extensive setup time thereby, limiting the accessibility for routine use. Additionally, the high cost and complexity of the devices restrict their widespread adoption.

Another well-known solution is Stryker Distal Targeting Jig. The targeting jig is first attached to the handle of nail inserting jig and aligned with the nail in the femur and then the pre-calibrated hole in the jig is aligned with the hole in the nail using an image intensifier. The surgeon uses this alignment to insert the distal locking screw with the help of a guiding sleeve. While the system improves accuracy, it still requires significant setup time, trained personnel, and a dependence on X-ray imaging, thus not solving the core problem of minimizing radiation exposure and time inefficiencies.

Despite the innovations, the advanced jigs and targeting systems are rarely seen in common use due to their complexity, high cost and the need for specialized training. As a result, the majority of patients still undergo the procedure using the traditional free-hand technique which continues to present challenges. The challenges include prolonged procedural times, exposure to radiation and the risk of bone damage due to multiple failed attempts. Therefore, there is a pressing need for further innovation in the field of orthopedic tools, specifically in the area of distal locking screw insertion to offer a solution that is faster, more precise and reduces the reliance on X-ray imaging while minimizing the risk of complications such as weakened bone areas from repeated errors.

### SUMMARY

The present invention relates to a medical tool, more specifically to a rapidil jig configured for dynamic locking of the nail inserted for fixation of fractured femur. The innovative tool eliminates the need for X-ray guidance during screw insertion, reducing radiation exposure to both the patient and the surgical team. The tool comprises sleeves and adjustable arms of varying lengths for improved stability and precision. The tool allows accurate screw placement with or without requiring guide wires or nail inserting jig handle for its fixation, improving efficiency and reducing complications. Adaptable to various femoral bone sizes, the rapidil jig ensures broad applicability and hence an ideal tool for both well-equipped medical centers and resource-limited settings. The innovative approach of the tool has the potential for wider use in orthopedic surgeries beyond distal femur fractures.

In an embodiment of the present invention, the invention discloses a rapid distal locking jig or rapidil jig for dynamic locking of the inserted intramedullary nail for bone fixation. The rapidil jig comprising an interconnecting bar extending between a proximal targeting arm, a distal targeting arm and a third arm. The proximal targeting arm of jig is configured for insertion of a proximal locking screw and the distal targeting arm of the jig is configured for the insertion of a distal locking screw. In the embodiment, a third arm is configured for insertion of a guide wire to maintain the jig in alignment during screw insertion. Further, the jig includes a plurality of sleeves which are inserted through each targeting arm. The plurality of sleeves comprises an inner headless sleeve configured to enter a pre-drilled hole in the bone for screw insertion, an outer sleeve configured to insert a locking screw into the drilled hole and a middle spacer sleeve positioned between the inner and the outer sleeves. The jig is configured to align the central axes of the targeting arms with the drilled holes in the femoral nail thus, allowing for the insertion of the locking screws.

In one of the embodiments of the present invention, the interconnecting bar ensures that the distance between the central axes of the targeting arms corresponds to the distance between the centres of the proximal and distal locking screw spaces in the pre-drilled holes of the femoral nail.

In one of the embodiments of the present invention, the proximal targeting arm is configured to maintain alignment during insertion of the proximal locking screw with the length of the arm optimized to rest firmly on the bone surface without tilting, if the jig is not stabilised with guide wire through the third arm.

In one of the embodiments of the present invention, the distal targeting arm has a shorter length than the proximal targeting arm thus, prevents tilting and ensuring that only the proximal arm contacts the bone surface.

In an embodiment of the present invention, the invention discloses a method for inserting locking screws into a femoral intramedullary nail. The method includes aligning the proximal round hole with a C-arm for imaging and securing the C-arm in place. In addition, a small incision is made in the skin in the area which appeared to be at the centre of the imaged hole and blunt dissection is performed to expose the bone cortex. In the embodiment, a first guide wire is positioned obliquely on the lateral cortex at the center of the planned proximal locking hole, followed by its insertion at right angle then the lateral cortex hole is drilled for the proximal locking screw. Further, the C-arm is adjusted to align the dynamic locking hole and a second guide wire is inserted into the lateral cortex which is followed by drilling, performed for the dynamic locking screw. The holes are made for positioning of the arms of alignment guide. A dynamic hole locator is used if any difficulty exists in positioning of alignment guide. An alignment guide is then inserted along the guide wires. After ensuring during screening of proper hole alignment, guide wires are inserted through medial cortex followed by drilling to create complete holes. The locking screws are inserted using a targeting jig and after removal of the jig, guide wires and screwdrivers the incisions are closed. Then, post-insertion, imaging is conducted to confirm the proper placement of the locking screws.

In one of the embodiments of the present invention, the alignment guide is configured for fine-tuning of alignment when round space is seen at the centre of the alignment ring with arrowheads before insertion of the guide wires within the planned holes (spaces “P” and “D”) in the distal femur to ensure precise alignment during insertion where “P” stands for a space for proximal hole and “D” stands for a dynamic screw hole space. In the embodiment, the hole “S” stands for a space for static screw hole which is adjacent to hole “D”.

In one of the embodiments of the present invention, the alignment guide includes an alignment ring with upper and lower solid segments, a mid-horizontal bar, a round gap and an arrowhead extension for precise alignment of the planned holes for locking screws with space ‘S’ of the dynamic hole which ultimately aligns the arms of the alignment guide with the planned distal locking holes through spaces P and D. After ensuring during screening a proper hole alignment guide wire are inserted through medica cortex followed by drilling to create complete holes.

In one of the embodiments of the present invention, the C-arm is used to provide real-time X-ray imaging for aligning the dynamic hole in the nail with the distal guide arm.

In one of the embodiments of the present invention, the targeting arms guide surgical instruments along planned screw holes and the sleeves maintain alignment and stability for precise insertion of locking screw into the femoral intramedullary nail.

For further clarification of the features and other embodiments of the invention, a more particular description is provided that will further explain the features and advantage of the invention with the illustration or the drawings. As will be appreciated, other embodiments of the present invention are possible utilizing, alone or in combination, one or more of the features set forth above or described in detail below.

### BRIEF DESCRIPTION OF THE DRAWINGS

A full understanding of the invention can be gained from the following description of the preferred embodiments when read in conjunction with the accompanying drawings in which:

FIG. 1A illustrates a perspective view of a rapidil jig with inserted sleeves, in accordance with the embodiments of the present invention disclosed herein.

FIG. 1B illustrates a perspective view of a rapidil jig, in accordance with the embodiments of the present invention disclosed herein.

FIG. 1C illustrates a perspective view of a plurality of sleeves of a rapidil jig, in accordance with the embodiments of the present invention disclosed herein.

FIG. 2A illustrates a perspective view during screening of alignment of the proximal hole of a nail at the centre of a ring (radiopaque) placed on the skin of distal thigh, in accordance with the embodiments of the present invention disclosed herein.

FIG. 2B illustrates a perspective view seen on the monitor of proximal hole centered within the radiopaque ring on screening, in accordance with the embodiments of the present invention disclosed herein.

FIG. 3A illustrates a perspective view of a guide wire positioned on lateral cortex in proximity of planned proximal locking hole, in accordance with the embodiments of the present invention disclosed herein.

FIG. 3B illustrates a perspective view of proper insertion of the guide wire through the hole, in accordance with the embodiments of the present invention disclosed herein.

FIG. 4A illustrates a perspective view of a first guide wire passing through the proximal locking hole of the distal nail, in accordance with the embodiments of the present invention disclosed herein.

FIG. 4B illustrates a perspective view of a second guide wire passing through the distal locking hole of the distal nail, in accordance with the embodiments of the present invention disclosed herein.

FIG. 5 illustrates a perspective view of a dynamic hole locator, in accordance with the embodiments of the present invention disclosed herein.

FIG. 6A illustrates a perspective view of an alignment guide prepared to be inserted through half holes of the nail, in accordance with the embodiments of the present invention disclosed herein.

FIG. 6B illustrates a perspective view of an alignment guide fully aligned to the proximal locking hole and distal locking hole, in accordance with the embodiments of the present invention disclosed herein.

FIG. 6C illustrates a perspective view of a central round hole of the alignment guide coinciding with the round space in the nail, in accordance with the embodiments of the present invention disclosed herein.

FIG. 6D illustrates a perspective view of insertion of guide wires through full thickness of the bone before creation of complete holes, in accordance with the embodiments of the present invention disclosed herein.

FIG. 6E illustrates a perspective view of complete holes created and aligned with the nail, in accordance with the embodiments of the present invention disclosed herein.

FIG. 7 illustrates a perspective view of the rapidil jig properly positioned before insertion of the screws accordance with the embodiments of the present invention disclosed herein.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION

References will now be made in detail to the presently preferred embodiments of the invention, examples of which are illustrated in the accompanying drawings. Throughout the following detailed description, the same reference numerals refer to the same elements in all figures.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and it is not intended to be limiting the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In the following description, reference will be made to the accompanying drawing, in which comparable functional elements are designated with like numerals. The aforementioned accompanying drawings show by way of illustration and not by the way of limitation, specific aspects and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in limited sense. It is noted that description herein is not intended as an extensive overview, and as such, concepts may be simplified in the interests of clarity and brevity. All documents mentioned in this application are hereby incorporated by reference in their entirety.

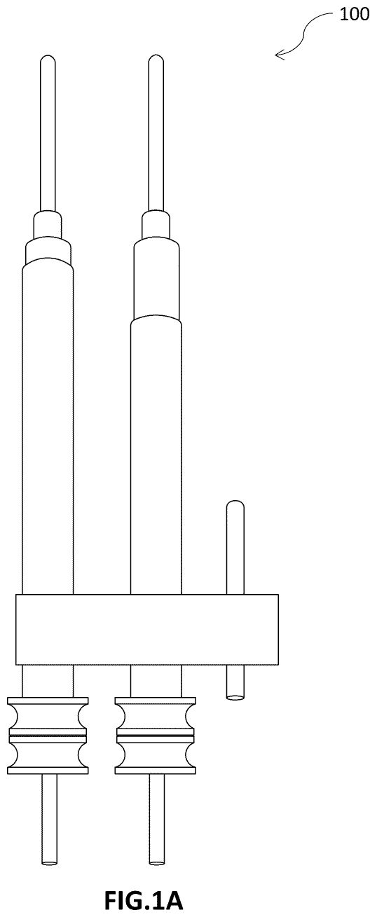

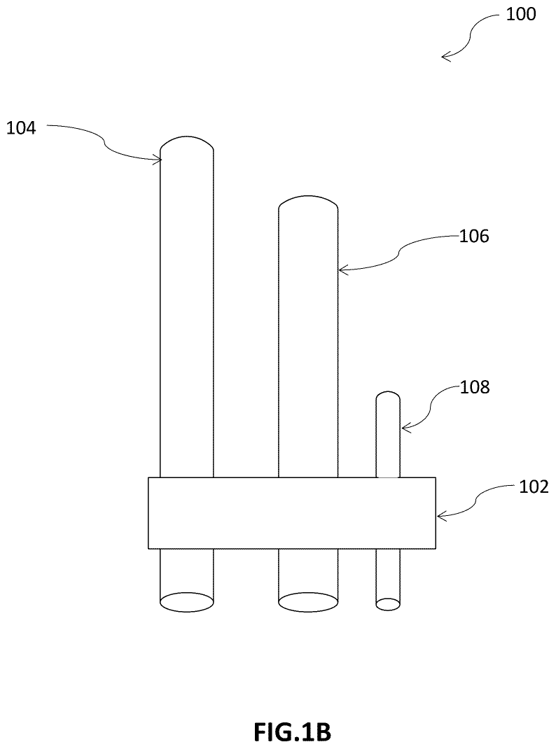

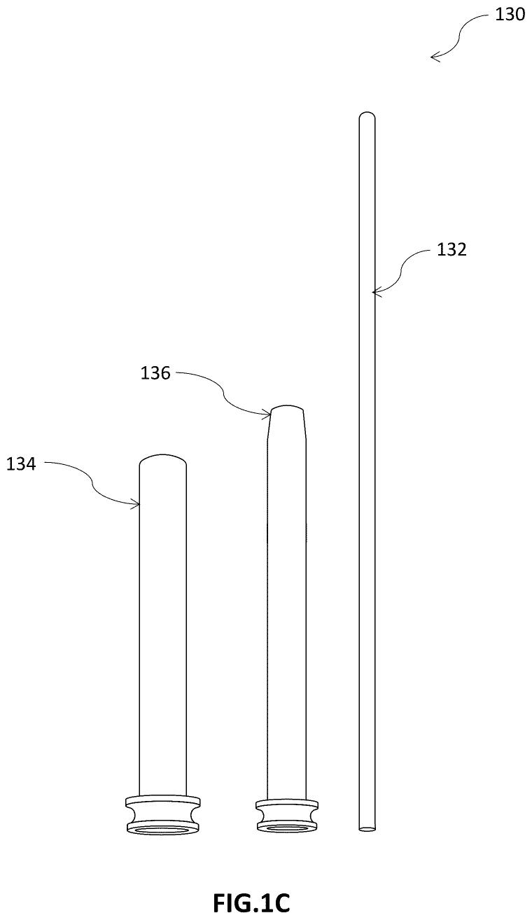

According to the embodiment of the present invention, a rapid distal locking jig 100 as shown in FIG. 1 is used for insertion of screws for dynamic locking during bone fixation. The jig 100 is configured to enhance the precision and efficiency of screw insertion in surgical operations, particularly when securing femoral nails. The jig 100 includes an interconnecting bar 102 that links a proximal targeting arm 104, a distal targeting arm 106 and a third arm 108. The interconnecting bar 102 plays a crucial role in ensuring that the central axes of the targeting arms are aligned with the drilled holes in the femoral nail. The precise alignment is essential for proper screw insertion. The jig's structure is configured to maintain a fixed distance between the central axes of the targeting arms which corresponds to the distance between the centers of the proximal and distal locking screw spaces in the femoral nail. This ensures that the jig 100 can facilitate the dynamic locking of screws without the need for adjustments during use, allowing for a more efficient procedure. In the embodiment, the rapidil jig 100 includes a plurality of sleeves 110 that are inserted through each targeting arm. The inner sleeve 112 is headless and is configured to enter the drilled hole in the bone thus, providing a clear path for the screw. The outer sleeve 114 is used for inserting the locking screw into the drilled hole, securing the bone fixation. Between these two sleeves is a middle spacer sleeve 116 which is configured to maintain the necessary distance between the inner 112 and outer sleeves 114, ensuring aligned position of the jig arms before the screws are inserted correctly and securely. The combined action of the sleeves allows for an efficient, streamlined process of locking screws into place. Additionally, the third arm 116 of the jig 100 is configured to hold a guide wire which helps maintain the jig's alignment during screw insertion particularly if the surgeon operates without assistant. The guide wire ensures that the jig remains firmly in place throughout the procedure, preventing any shifting that could lead to misalignment and improper screw placement. Further, the jig 100 is configured to avoid tilting during use. The proximal targeting arm 104 is optimized in length and configured to rest firmly on the surface of the bone without tilting, ensuring stable support during the insertion of the proximal locking screw. The distal targeting arm 106 is shorter than the proximal arm 104 which prevents any tilting during the procedure, ensuring that only the proximal arm contacts the bone surface. The innovative jig 100 contributes to the overall stability and precision, making the jig 100 valuable tool for surgeons performing bone fixation operations.

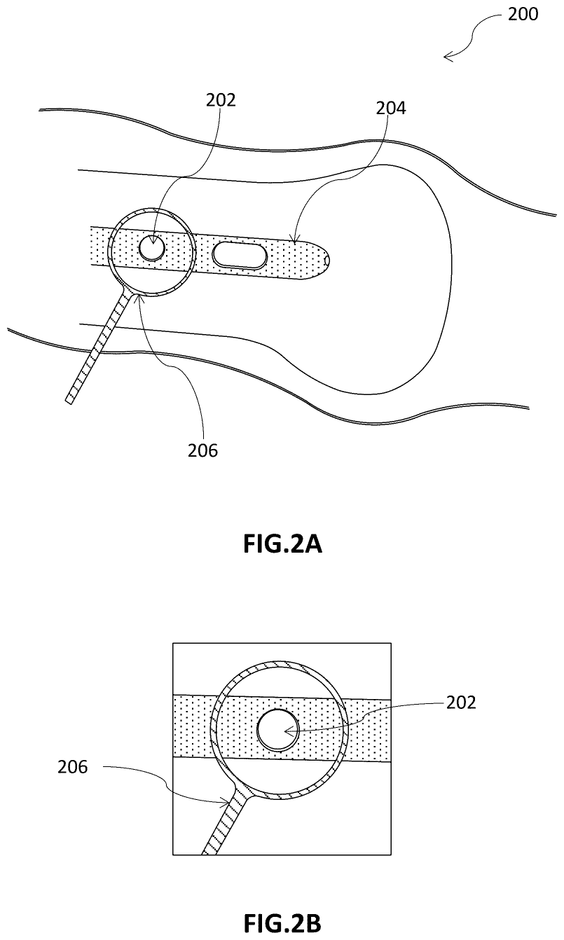

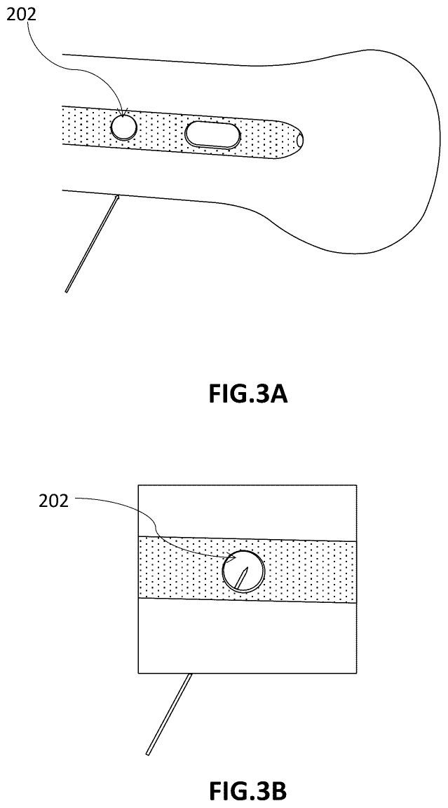

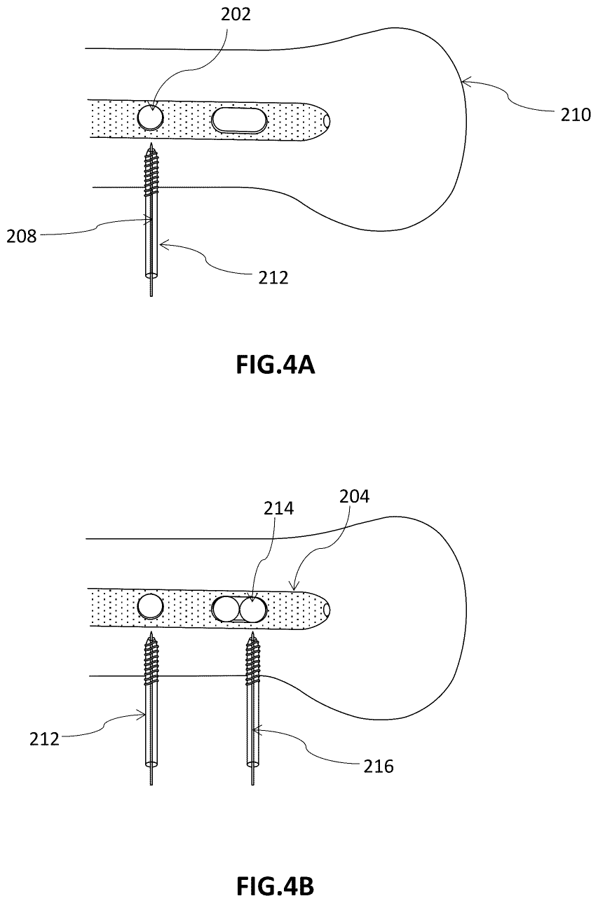

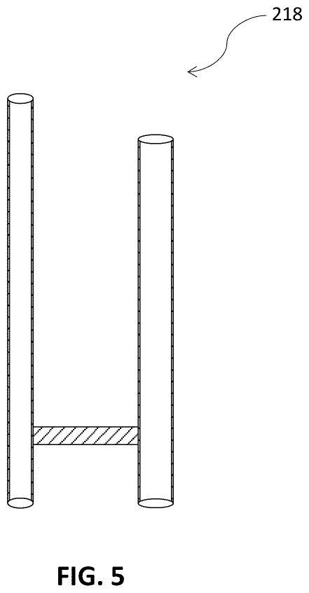

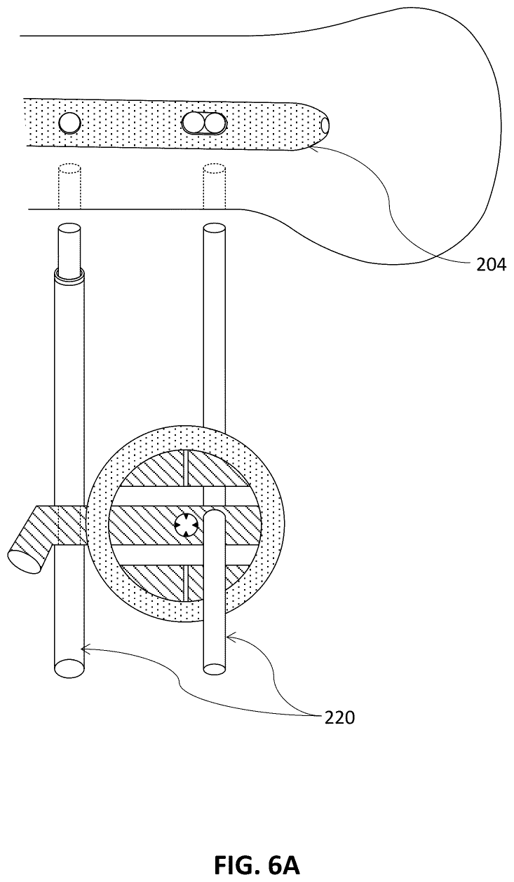

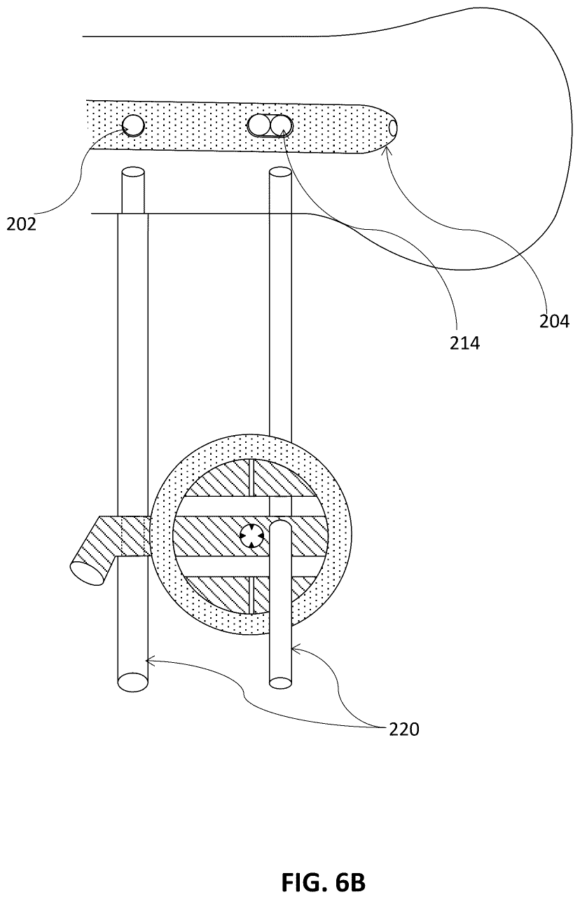

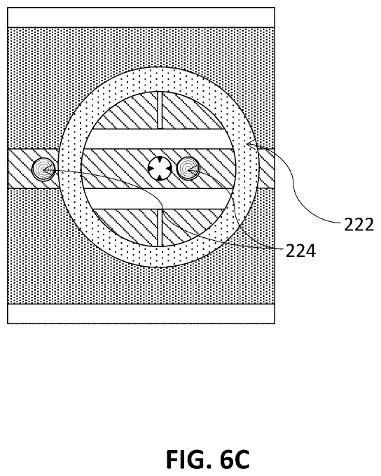

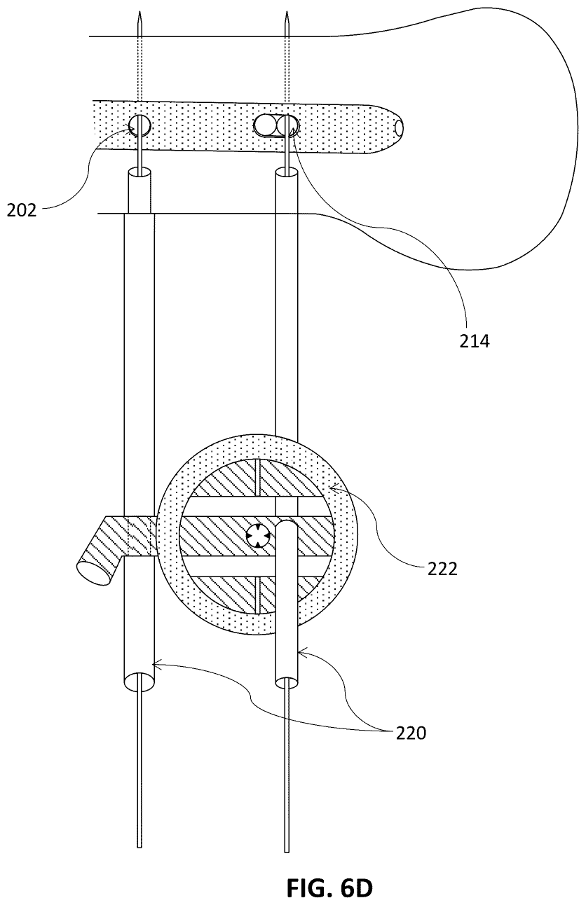

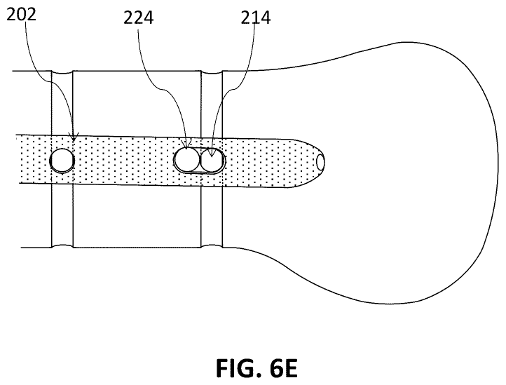

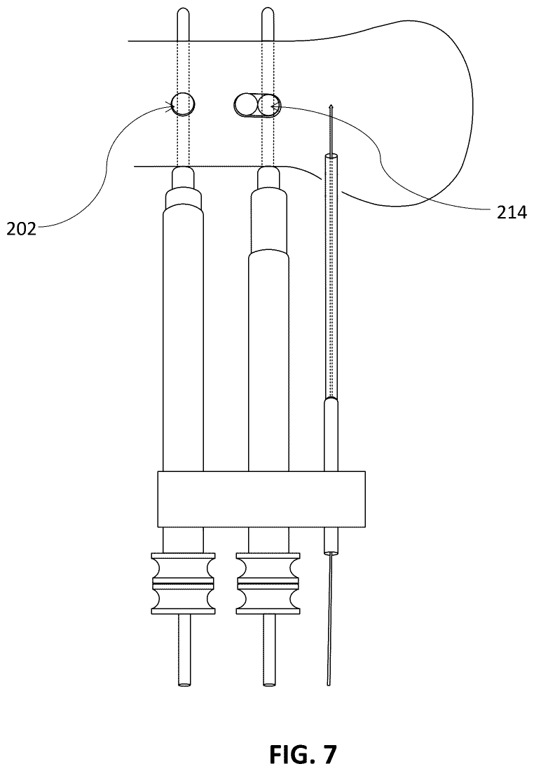

According to the embodiment of the present invention, the method for inserting locking screws into a femoral intramedullary nail process is disclosed as shown by FIG. 2-FIG. 7. In the embodiment, according to FIG. 2A, the method 200 begins by aligning the proximal round hole 202 (space “P”) of the distal nail 204 using a C-arm for imaging. The C-arm is precisely positioned and not moved so that the round hole 202 is centred. A radiopaque ring is positioned on the skin in the proximity of the round hole 202. In the embodiment, the FIG. 2B discloses the radiographic imaging is used to confirm that the round hole 202 is accurately positioned at the centre of the radiopaque ring 206. Screening is stopped and a small incision is made on the skin at the center of the radiopaque ring in the line of round hole 202 in the nail and blunt dissection is performed to reach the bone cortex. In the embodiment, according to FIG. 3A-FIG. 3B, the first guide wire is placed obliquely through the soft tissue after the incision to position its tip on the lateral cortex at the point which appears to be at the centre of the round hole 202. As shown in FIG. 4, the first guide wire 208 is then inserted at the right angles through the outer cortex of the femur 210 through the center of the proximal locking hole 202. Drilling is carried out along the first guide wire 208 with a cannulated drill bit 212 to create the hole through the lateral cortex for the planned proximal locking screw. According to FIG. 4B, the C-arm is reconfigured to align the dynamic locking hole 214 (space “D”) of the nail 204. The second guide wire 216 is inserted through the lateral cortex into the dynamic locking hole 214. Drilling along the second guide wire 216 using the cannulated drill bit 212 creates the hole through the lateral cortex for the planned dynamic locking screw. In the embodiment, according to FIG. 5, if misalignment is detected between the holes, a dynamic hole locator 218 is employed to correct the discrepancy. The dynamic hole locator 218 ensures that the holes for the screws are properly aligned, facilitating accurate screw placement. In the embodiment, according to FIG. 6A, an alignment guide 220 is prepared to be inserted in the half holes of the nail 204. Additionally, the FIG. 6B discloses the alignment guide 220 are aligned properly in the holes i.e. proximal locking hole 202 and the dynamic locking hole 214. The alignment guide is moved to align space ‘S’ of dynamic hole of the nail 204 with the central hole in the alignment guide ‘C’ 220. In the embodiment, according to FIG. 6C, an alignment ring 222 discloses the central round hole coinciding with the round space ‘S’ 224 in the nail 204. This ensures that the proper alignment is reached. In the embodiment, according to FIG. 6D, the first guide wire is inserted through the proximal hole 202 and the second guide wire is inserted through the dynamic locking hole 214. The guide's 220 role is crucial in maintaining the exact alignment while drilling, ensuring that the partial holes are extended into complete holes for the insertion of locking screws. Further, the alignment guide 220 has alignment ring 222. The alignment ring 222 is characterized by 18 mm outer diameter and a 4 mm thick rim. The ring 222 includes upper and lower solid segments each 3 mm thick, positioned at a vertical distance of 10 mm from the centre of the ring 222 one on each side. The segment has a 2 mm wide vertical gap aligned with the centre of the ring 222 which helps in accurate vertical alignment. A horizontal bar 10 mm wide runs along the horizontal diameter of the ring 222 and connects the two arms of the guide 220. The ring 222 includes a circular gap with a 5 mm diameter in the horizontal bar, positioned proximally to the distal arm of the guide 220. The distance between the centre of this gap and the central axis of the distal arm corresponds to the distance between centre of spaces “S” and “D” of the holes in the nail 204, ensuring accurate alignment. Additionally, four 1 mm long arrowhead-like extensions are placed at the ends of the vertical and horizontal diameters of the round gap. The extensions are positioned at the 3, 6, 9 and 12 o'clock positions and functions as aiming reticles to guide the surgeon in achieving precise alignment. The surgeon holds the alignment guide 220 with a handle attached to the proximal arm with one hand while manipulating a guide wire with the other using tools such as a K-wire driver or guide wire holder to secure the wire. Further, alignment of the guide 220 with the planned locking screw holes is confirmed through imaging showing the arrowheads at 3, 6, 9, and 12 o'clock positions then full alignment is achieved. Further, the FIG. 6E discloses holes which are ready and are properly positioned. The hole spaces are represented as ‘P’ 202, ‘S’ 224 and ‘D’ 214 respectively. In the embodiment, according to FIG. 7, a targeting jig is applied to align the screw insertion points. The jig is equipped with several sleeves to facilitate the correct alignment of targeting arms for placement of the screws. The jig aligns the central axes of its targeting arms with the drilled holes in the femoral nail, allowing for the insertion of the locking screws with precise accuracy. The screws are inserted first through the proximal locking hole 202 and then through the distal locking hole 214. The screw driver is not removed after insertion of first screw. The second screw i.e. the dynamic locking screw is inserted with longer screw driver. If necessary, a guide wire can be used to stabilize the jig, especially if the surgeon is working without an assistant. The guide wire is inserted through thin sleeves, which has been passed through the third arm of the jig and stabilize the jig with the aligned targeting arms. Once both screws are inserted and tightened, the targeting jig and other accessories such as the guide wires and screwdrivers are removed.

In one of the embodiments of the present invention, the spaces allocated for different holes are as follows: “P” represents the space for the proximal locking screw, “S” corresponds to the space for the static locking screw and “D” refers to the space for the dynamic locking screw.

In one embodiment of the present invention, the alignment guide includes two parallel cylindrical arms connected by an interconnecting part. The outer diameter matches the diameter of the screw holes and the inner diameter fits the guide wire thickness.

In one of the embodiments of the present invention, the C-arm is used to provide real-time X-ray imaging for aligning the locking screw holes with the guide arms.

In one of the embodiments of the present invention, the targeting arms guide surgical instruments along planned screw holes which help in completing the lateral cortex holes to full thickness holes in the bone.

In one of the embodiments of the present invention, the diameter of drilled hole is 4 mm and the diameter of spaces S, P and D is 5 mm.

It should be understood that the examples provided herein are intended only for purposes of illustration and any number of other implementations is also contemplated. Additionally, the referenced examples (including the described rules and/or other techniques) can be combined in any number of ways.

Although an overview of the inventive subject matter has been described with reference to specific example implementations, various modifications and changes can be made to those implementations without departing from the broader scopes of implementation of the present disclosure. Such implementation of the inventive subject matter can be referred to herein, individually or collectively, by the term “invention” merely for convenience without intending to voluntarily limit the scope of this application to any single disclosure or inventive concept if more than one is, in fact is disclosed.

The implementations illustrated herein are described in sufficient detail to enable those skilled in the art to practice the teachings disclosed. Other implementations can be used and derived therefrom, such that structural substitutions and changes can be made without departing from the scope of this disclosure. The Detailed Description, therefore, is not to be taken in a limiting sense, and the scope of various implementations is defined only by the appended claims, along with the full range of equivalents to which such claims are entitled.

As used herein, the term “or” can be construed in either an inclusive or exclusive sense. Moreover, plural instances can be provided for resources or structures described herein as a single instance. These and other variations, modifications, additions, and improvements fall within a scope of implementations of the present disclosure as represented by the appended claims. The specification and drawings are, accordingly, to be regarded in an illustrative rather than a restrictive sense.

What is claimed is:

1. A rapid distal locking jig for insertion of locking screws through distal holes in the nail for bone, the rapid distal locking jig comprising:
an interconnecting bar extending between a proximal targeting arm, a distal targeting arm and a third arm; and
a plurality of sleeves which are parallel to each other;
the proximal targeting arm configured for insertion of a proximal locking screw; and
the distal targeting arm configured for the insertion of a distal locking screw;
the third arm configured for insertion of a guide wire to maintain the jig in alignment during screw insertion;
wherein the plurality of sleeves are inserted through the proximal targeting arm and the distal targeting arm,
wherein the plurality of sleeves comprising:
an inner headless sleeve configured to enter a drilled holes in the bone for screw insertion;
an outer sleeve configured to insert a locking screw into the drilled hole;
a middle spacer sleeve positioned between the inner and the outer sleeves;
wherein the jig aligns the central axes of the targeting arms with the drilled holes passing through the femoral nail, for allowing the insertion of the locking screws.

2. The rapid distal locking jig as claimed in claim 1, wherein the interconnecting bar ensures that the distance between the central axes of the targeting arms corresponds to the distance between the centres of the proximal and distal locking screw spaces in the pre-drilled holes of the femoral nail.

3. The rapid distal locking jig as claimed in claim 1, wherein the proximal targeting arm is configured to maintain alignment during insertion of the proximal locking screw with the length of the arm optimized to rest firmly on the bone surface without tilting.

4. The rapid distal locking jig as claimed in claim 1, wherein the distal targeting arm has a shorter length than the proximal targeting arm and prevents tilting when placed on the bone, ensuring only the proximal targeting arm rests on the bone surface.

5. A method of insertion of locking screws through distal end of a nail before completion of intra-medullary nailing of femur, the method comprising the steps of:
(a) aligning the proximal round hole(space “P”) of the distal nail with a C-arm for imaging and securing the C-arm in place;
(b) incising a small opening on the skin looking to be at the centre of the imaged round hole and performing blunt dissection to expose the bone cortex;
(c) positioning tip of a first guide wire obliquely on the lateral cortex looking to be at the center of the space “P” and inserting at right angle;
(d) drilling along the first guide wire using cannulated drill bit to create a hole through the lateral cortex for the planned proximal locking screw;
(e) configuring the C-arm to align the dynamic locking hole (space “D”) of the nail;
(f) inserting a second guide wire through the lateral cortex into the dynamic locking hole (space “D”) and repeating steps from (a) to (e);
(g) drilling along the second guide wire to create a hole through the lateral cortex for the planned dynamic locking screw;
(h) configuring a dynamic hole locator to correct any misalignment between the holes;
(i) inserting an alignment guide along the two guide wires in the lateral cortex holes after screening and maneuvering proper alignment of the holes of distal nail in the femur with the holes in the guide arms is achieved then guide wires are pushed through full thickness of the bone in line of the planned holes;
(j) drilling along the guide wires to convert partial holes into complete holes for the planned locking screws;
(k) inserting the locking screws into the screw holes;
(l) performing post-insertion imaging to confirm the placement of the locking screws.

6. The method as claimed in claim 5, wherein the alignment guide is configured for fine-tuning the position of the guide wires within the planned holes (spaces “P” and “D”) in the distal femur to ensure precise alignment during insertion.

7. The method as claimed in claim 5, wherein the alignment guide includes an alignment ring with upper and lower solid segments, a mid-horizontal bar, a round gap and arrowhead extensions for precise alignment of the planned holes of the nail with the guide arms, and along mid vertical line small thin gap in each solid segment.

8. The method as claimed in claim 5, wherein the locator assures the proper positioning of the dynamic hole to ensure positioning of the two arms of alignment guide in the lateral cortex holes.

9. The method as claimed in claim 5, wherein the C-arm is used to provide real-time X-ray imaging for aligning the locking screw holes with the guide arms.

10. The method as claimed in claim 5, wherein the targeting arms guide surgical instruments along planned screw holes, and the sleeves maintain alignment and stability for precise insertion of locking screw into the femoral intramedullary nail.

11. The method as claimed in claim 5, wherein the spaces allocated to different holes are P (the space for proximal locking screw), S (the space for static locking screw) and D (the space for dynamic locking screw).
